(19) United States
(12) Patent Application Publication
(10) Pub. No.: US 2026/0225991 A1
(43) Pub. Date: Aug. 6, 2026

(54) EMULSIFIERS FORMED FROM POLYOL ESTERS OF FATTY ACIDS

(71) Applicant: Ruby Bio Inc., San Carlos, CA (US)

(72) Inventors: Pavan Kumar Reddy Kambam, Sunnyvale, CA (US); Denis Kembero Ontumi, Dublin, CA (US); Charles Scott Silver, Oakland, CA (US)

(21) Appl. No.: 18/869,027

(22) PCT Filed: May 26, 2023
(86) PCT No.: PCT/US2023/067583

§ 371 Date: Nov. 25, 2024

Related U.S. Application Data
(60) Provisional application No. 63/365,404, filed on May 26, 2022.
(60) Provisional application No. 63/365,405, filed on May 26, 2022.
(60) Provisional application No. 63/365,406, filed on May 26, 2022.

(51) Int. Cl. C07C 69/33; A23L 2/52; A23L 29/10; A61K 8/06; A61K 8/37; A61Q 1/14; A61Q 5/12; A61Q 19/00; C09D 7/63; C09J 11/06; C11D 1/74

(52) U.S. Cl. CPC C07C 69/33; A23L 2/52; A23L 29/10; A61K 8/062; A61K 8/375; A61Q 1/14; A61Q 5/12; A61Q 19/007; C09D 7/63; C09J 11/06; C11D 1/74; A61K 2800/33

(57) ABSTRACT

An emulsifier, a water-in-oil (W/O) emulsion composition, and oil-in-water (O/W) emulsion composition each including a polyol lipid having a polyol ester of fatly acid (PEFA) compound.

### REFERENCE TO RELATED APPLICATIONS

This application claims priority to U.S. Provisional Patent Application Ser. No. 63/365,404, entitled Compounds, Compositions, and Methods of Use Thereof, filed May 26, 2022; U.S. Provisional Patent Application Ser. No. 63/365,405, entitled Polyol Ester of Fatty Acids (PEFAs) as Antifoam, filed May 26, 2022; and U.S. Provisional Patent Application Ser. No. 63/365,406, entitled Polyol Ester of Fatty Acids (PEFAs) as Emulsifiers, filed May 26, 2022; and hereby incorporates the provisional applications by reference herein in their entireties.

### TECHNICAL FIELD

The present disclosure relates to emulsifiers; and water-in-oil emulsion compositions and oil-in-water emulsion compositions having polyol lipids formed from polyol esters of fatty acids (PEFAs) compounds.

### BACKGROUND

Emulsifiers for water-in-oil emulsions exhibit better moisture resistance than oil-in-water emulsions. However, stabilizing water-in-oil emulsions is more difficult than stabilizing oil-in-water emulsions. In addition, water-in-oil emulsions often suffer problems for emulsification/dispersion properties (e.g., difficulty to stabilize an emulsion while maintaining a low emulsion viscosity, and difficulty in maintaining stability in regions having wide temperature variations), and such water-in-oil emulsions have difficulty producing a soft feeling to touch, light smoothness and good spreadability and good moisture retention. These problems for water-in-oil emulsions are an issue when formulating cosmetics compositions.

In food processing, a mixture of food components is typically subjected to a range of thermal and mechanical treatments, for example, baking, boiling, steaming, freezing, kneading, mixing, and extruding, to form a food product with the desired taste, texture, as well as desired visual properties such as color and shape. Food components may be incompatible with other food components when present in a mixture. For example, oil and water are immiscible, and form a thermodynamically unstable emulsion when mixed together. The oil and water phases will gradually separate, causing the emulsion to coalesce, flocculate, cream or break. This can negatively impact the characteristics of a food product, such as shelf-life, taste, or aesthetics.

An example of an emulsifier used for beverages is gum Acacia (gum Arabic) prepared from an exudate from the stems and branches of the sub-Saharan species of the Acacia tree, Acacia senegal and Acacia seyal. Due to its high demand and unreliable supply, gum Arabic can be difficult and expensive to obtain.

Therefore, there is a need for developing renewable or biodegradable alternative emulsifiers, water-in-oil (W/O) emulsion compositions, and oil-in-water (O/W) emulsion compositions having emulsion stability over time, when subjected to heat, or that can improve the constitution of an end consumer product.

### BRIEF DESCRIPTION OF THE DRAWINGS

FIG. 1 is a photo depicting a first, left vial with ZnO particles added to an emulsifier according to one embodiment and a second, right vial with ZnO particles added to a conventional emulsifier, both vials having been agitated.

FIG. 2 is a photo depicting the vials of FIG. 1, both vials having been stored at 60° C. for 1 week after agitation.

### DETAILED DESCRIPTION

The details of various embodiments of the invention are set forth in the description below. Other features, objects, and advantages of the invention will be apparent from the description and the drawings, and from the claims.

### Definitions

As used herein, a “foodstuff” refers to material that is edible or drinkable, or a material that can be processed into an edible or drinkable material.

As used herein, the term “cosmetic composition” refers to a product to be applied to human skin, nails, hair, and the like, and is used for beauty purposes.

It will be appreciated, however, the term “composition” refers to cosmetic compositions and other applications, including pesticides, paints, coatings, inks, adhesives, among others.

As used herein, weight percent (wt %), percent by weight, % by weight, and the like are synonyms that refer to the concentration of a substance as the weight of that substance divided by the weight of the composition and multiplied by 100.

The term “about,” preceding a figure encompasses plus or minus 10%, or less, of the value of said figure. It is to be understood that the value to which the term “about” refers is itself also specifically, and preferably, disclosed.

The term “alkyl” refers to a straight or branched alkyl group. Exemplary alkyl groups are methyl, ethyl, propyl, isopropyl, butyl, isobutyl, and tert-butyl.

The term “halogen” means F, Cl, Br, or I.

As used herein, the term “salt or acid thereof” refers to any salts or acids of PEFA compounds. Exemplary salts and acids of PEFA compounds are suitable for use in contact with the tissues of humans and lower animals without undue toxicity, irritation, allergic response and the like, and are commensurate with a reasonable benefit/risk ratio. Salts can include pharmaceutically or biologically acceptable salts. Likewise, acids can include pharmaceutically or biologically acceptable acids. Pharmaceutically or biologically acceptable salts and acids are well known in the art. For example, S. M. Berge et alt, describe pharmaceutically or biologically acceptable salts in detail in J. Pharmaceutical Sciences, 1977, 66, 1-19, incorporated herein by reference. Pharmaceutically or biologically acceptable salts of the compounds of this disclosure can include those derived from suitable inorganic and organic acids and bases, while pharmaceutically or biologically acceptable acids of the compounds of this disclosure can include suitable inorganic and organic acids. Examples of pharmaceutically or biologically acceptable inorganic acids can include hydrochloric acid, hydrobromic acid, phosphoric acid, sulfuric acid, nitric acid, and perchloric acid or with organic acids such as acetic acid, oxalic acid, maleic acid, tartaric acid, citric acid, succinic acid, propionic acid or malonic acid or by using other methods used in the art such as ion exchange. Examples of pharmaceutically or biologically acceptable acid addition salts are salts of an amino group formed with such examples of inorganic acids. Another example of a pharmaceutically or biologically acceptable acid is a carboxylic acid, where the carboxylated version of the PEFA compound includes a carboxyl group. Other pharmaceutically or biologically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically or biologically acceptable salts include, when appropriate, ammonium, quaternary ammonium, and amine cations formed using counterions such as halide, hydroxide, carboxylate, sulfate, phosphate, nitrate, lower alkyl sulfonate and aryl sulfonate.

The present disclosure includes both the (R) and (S) configuration at each stereo center, even in cases where drawn as defined or drawn undefined. Additionally, the present disclosure includes racemic compositions of compounds disclosed herein. The present disclosure includes scalemic compositions of compounds disclosed herein. The present disclosure includes enantioenriched compositions of compounds disclosed herein.

Polyol lipid biosurfactants as described herein can include polyol esters of fatty acids (PEFAs).

Polyol lipids can be isolated and/or purified from a yeast culture according to methods known in the art. Since the polyol lipids are extracellularly secreted from yeast cells, the one or more polyol lipids can be isolated and/or purified from the yeast cell culture without cell lysis or extraction requiring an organic solvent.

Polyol esters of fatty acids are amphiphilic molecules comprising a sugar alcohol, e.g., a D-mannitol and/or a D-arabitol, esterified to the carboxyl end of a 3-hydroxy fatty acyl moiety, which may or may not be acetylated. The non-esterified hydroxy groups of the sugar alcohol may or may not be acetylated as well. In some embodiments, the one or more polyol lipids produced are a mixture of similar compounds containing (R)-3-hydroxy fatty acyl moieties with varying chain lengths, in the range of about 8 to 24 carbons, preferably in the range between 12 to 20 carbons. In some embodiments, the (R)-3-hydroxy fatty acyl moieties can present varying degrees of unsaturation in the range of about 0 to 6, e.g., in the range between 2 to 5. In some embodiments, the non-esterified hydroxy groups of the sugar alcohol can be esterified to acetyl groups. In some embodiments the sugar alcohol can be fully acetylated. In some embodiments, the sugar alcohol can be non-acetylated. In some embodiments, acetylations can range between these two states.

In some embodiments, a polyol lipid disclosed herein is an acetylated C12:0-3-hydroxy fatty acid esterified to D-arabitol with 3 acetylations. In some embodiments, a polyol lipid disclosed herein is an acetylated C14:0-3-hydroxy fatty acid esterified to D-mannitol with 4 acetylations. In some embodiments, a polyol lipid disclosed herein is an acetylated C16:0-3-hydroxy fatty acid esterified to D-mannitol with 2 acetylations. In some embodiments, a polyol lipid disclosed herein is an acetylated C16:0-3-hydroxy fatty acid esterified to D-arabitol with 2 acetylations. In some embodiments, a polyol lipid disclosed herein is an acetylated C14:0-3-hydroxy fatty acid esterified to D-arabitol with 4 acetylations. In some embodiments, a polyol lipid disclosed herein is an acetylated C14:0-3-hydroxy fatty acid esterified to D-mannitol with 5 acetylations. In some embodiments, a polyol lipid disclosed herein is an acetylated C16:0-3-hydroxy fatty acid esterified to D-mannitol with 3 acetylations. In some embodiments, a polyol lipid disclosed herein is an acetylated C16:0-3-hydroxy fatty acid esterified to D-arabitol with 3 acetylations. In some embodiments, a polyol lipid disclosed herein is an acetylated C16:0-3-hydroxy fatty acid esterified to D-mannitol with 4 acetylations. In some embodiments, a polyol lipid disclosed herein is an acetylated C18:0-3-hydroxy fatty acid esterified to D-mannitol with 2 acetylations. In some embodiments, a polyol lipid disclosed herein is an acetylated C16:0-3-hydroxy fatty acid esterified to D-mannitol with 5 acetylations. In some embodiments, a polyol lipid disclosed herein is an acetylated C16:0-3-hydroxy fatty acid esterified to D-mannitol with 4 acetylations. In some embodiments, the polyol lipid is an acetylated C18:0-3-hydroxy fatty acid esterified to D-mannitol with 2 acetylations. In some embodiments, a polyol lipid disclosed herein is an acetylated C16:0-3-hydroxy fatty acid esterified to D-mannitol with 5 acetylations. In some embodiments, a polyol lipid disclosed herein is an acetylated C16:0-3-hydroxy fatty acid esterified to D-arabitol with 4 acetylations. In some embodiments, a polyol lipid disclosed herein is an acetylated C18:0-3-hydroxy fatty acid esterified to D-mannitol with 3 acetylations. In some embodiments, a polyol lipid disclosed herein is an acetylated C18:0-3-hydroxy fatty acid esterified to D-mannitol with 4 acetylations. In some embodiments, a polyol lipid disclosed herein is an acetylated C18:0-3-hydroxy fatty acid esterified to D-arabitol with 3 acetylations. In some embodiments, a polyol lipid disclosed herein is an acetylated C18:0-3-hydroxy fatty acid esterified to n-mannitol with 5 acetylations. In some embodiments, a polyol lipid disclosed herein is an acetylated C18:0-3-hydroxy fatty acid esterified to D-arabitol with 4 acetylations. In some embodiments, a polyol lipid disclosed herein is an acetylated C20:0-3-hydroxy fatty acid esterified to D-mannitol with 3 acetylations. In some embodiments, a polyol lipid disclosed herein is an acetylated C20:0-3-hydroxy fatty acid esterified to D-mannitol with 4 acetylations.

### Exemplary Compounds

In some embodiments, a polyol lipid is a PEFA compound represented by a compound selected from the group consisting of:

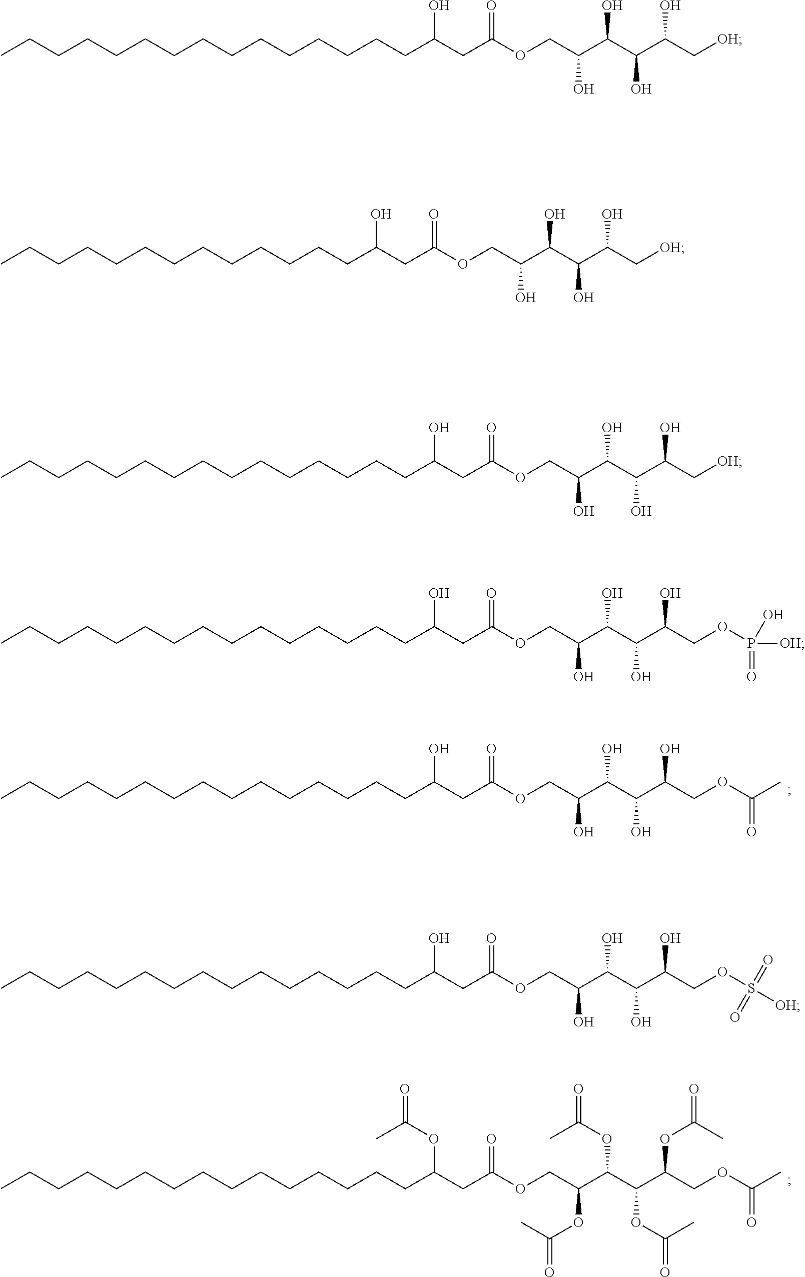

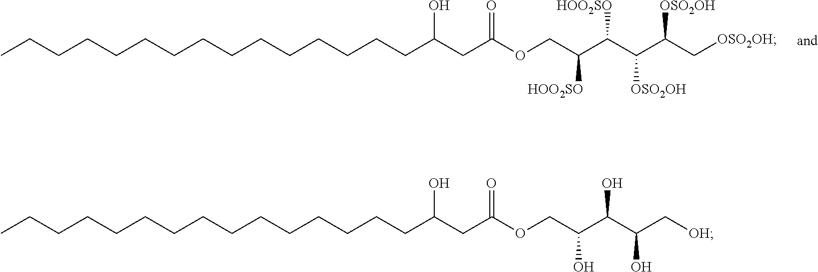

or salt or acid thereof.

In some embodiments, a polyol lipid is a PEFA compound represented by Formula (I) or (II):

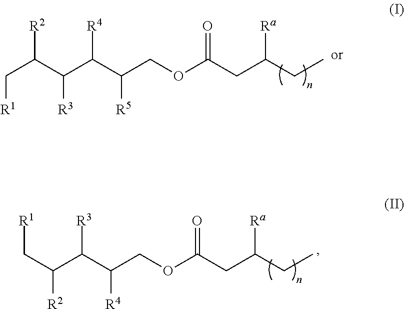

or salt or acid thereof,
wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-20;
j is 2-12;
k is 4-26;
m is 1-16; and each p is independently selected from 1-10.

In some embodiments, a polyol lipid is a PEFA compound of Formula (I) or (II):

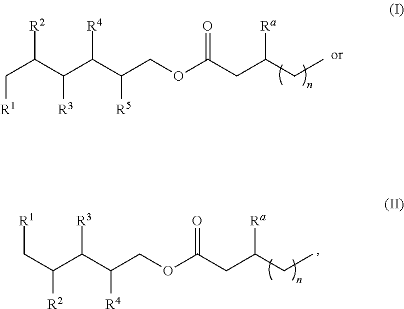

or salt or acid thereof,
wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-20;
j is 2-12;
k is 4-26;
m is 1-16; and
each p is independently selected from 1-10,
wherein
   when n is 12 or 14 and R¹ is —OH or —(O)Ac, then R^{a} is not —(O)Ac or —(O)Me;
   when n is 12 and R^{a} is —OH, then R² is not —OH or;
   when n is 12 or 14, R^{a} is —OH, R² is OH, R³ is OH, and R⁴ is OH, then R¹ is (O)R^{x} or —(O) (C₂H₅O)ₚR^{y};
   when n is 12 or 14, R¹ is —(O)S(O₂)OH, R² is —(O)S(O₂)OH, R³ is —(O)S(O₂)OH, R⁴ is —(O)S(O₂)OH, and R⁵ is —(O)S(O₂)OH, then R^{a} is —(O)C(O)R^{x}, (O)R^{x}, —(O)C(O)OR^{x}, —(O)C(O)N(H)R^{x}, —(O)(C₂H₅O)ₚR^{y}, —(O)P(O)(OH)₂, or —(O)S(O₂)OH.

In some embodiments, a compound of Formula (I) or (II), wherein
R¹ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R² is selected from the group consisting of —OH, —(O)C(O)R^{x}, —(O)R^{x}, —(O)C(O)OR^{y}, —(O)C(O)N(H)R^{y}, —(O) (C₂H₅O)ₚR^{y}, —(O)P(O)(OH)₂, and —(O)S(O₂)OH;
R³ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R⁴ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R⁵ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R^{a} is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-11; and
each p is independently selected from 1-10.

In some embodiments, a polyol lipid is a PEFA compound represented by Formula (I-a) or (II-a):

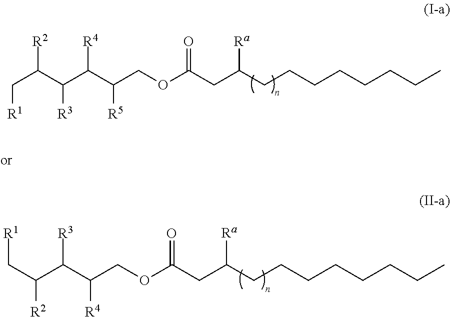

or salt or acid thereof,
wherein
n is 6-12.

### R¹

In some embodiments, R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ), where —(CⱼHₖOₘ) can be in linear or cyclic form. In some embodiments, R¹ is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R¹ is —H. In some embodiments, R¹ is —P(O)(OH)₂, or —S(O₂)OH. In some embodiments, R¹ is —C(O)Rₓ. In some embodiments, R¹ is —Ac. In some embodiments, R¹ is —R^{x}. In some embodiments, R¹ is —C(O)OR^{y}. In some embodiments, R¹ is —C(O)N(H)R^{y}. In some embodiments, R¹ is —(C₂H₅O)ₚR^{y}. In some embodiments, R¹ is —P(O)(OH)₂. In some embodiments, R¹ is —S(O₂)OH. In some embodiments, R¹ is —(CⱼHₖOₘ).

### R²

In some embodiments, R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ), where —(CⱼHₖOₘ) can be in linear or cyclic form. In some embodiments, R² is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R² is —H. In some embodiments, R² is —P(O)(OH)₂, or —S(O₂)OH. In some embodiments, R² is —C(O)R^{x}. In some embodiments, R² is —Ac. In some embodiments, R² is —R^{x}. In some embodiments, R² is —C(O)OR^{y}. In some embodiments, R² is —C(O)N(H)R^{y}. In some embodiments, R² is —(C₂H₅O)ₚR^{y}. In some embodiments, R² is —P(O)(OH)₂. In some embodiments, R² is —S(O₂)OH. In some embodiments, R² is —(CⱼHₖOₘ).

### R³

In some embodiments, R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ), where —(CⱼHₖOₘ) can be in linear or cyclic form. In some embodiments, R³ is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R³ is —H. In some embodiments, R³ is —P(O)(OH)₂, or —S(O₂)OH. In some embodiments, R³ is —C(O)R^{x}. In some embodiments, R³ is —Ac. In some embodiments, R³ is —R^{x}. In some embodiments, R³ is —C(O)OR^{y}. In some embodiments, R³ is —C(O)N(H)R^{y}. In some embodiments, R³ is —(C₂H₅O)ₚR^{y}. In some embodiments, R³ is —P(O)(OH)₂. In some embodiments, R³ is —S(O₂)OH. In some embodiments, R³ is —(CⱼHₖOₘ).

### R⁴

In some embodiments, R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ), where —(CⱼHₖOₘ) can be in linear or cyclic form. In some embodiments, R⁴ is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R⁴ is —H. In some embodiments, R⁴ is —P(O)(OH)₂, or —S(O₂)OH. In some embodiments, R⁴ is —C(O)R^{x}. In some embodiments, R⁴ is —Ac. In some embodiments, R⁴ is —R^{x}. In some embodiments, R⁴ is —C(O)OR^{y}. In some embodiments, R⁴ is —C(O)N(H)R^{y}. In some embodiments, R⁴ is —(C₂H₅O)ₚR^{y}. In some embodiments, R⁴ is —P(O)(OH)₂. In some embodiments, R⁴ is —S(O₂)OH. In some embodiments, R⁴ is —(CⱼHₖOₘ).

### R⁵

In some embodiments, R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ), where —(CⱼHₖOₘ) can be in linear or cyclic form. In some embodiments, R⁵ is selected from the group consisting of —C(O)R^{x}, —R^{x}, —C(O)OR^{x}, —C(O)N(H)R^{y}, —(C₂H₅O)ₚR^{y}, —P(O)(OH)₂, and —S(O₂)OH. In some embodiments, R⁵ is —H. In some embodiments, R⁵ is —P(O)(OH)₂, or —S(O₂)OH. In some embodiments, R⁵ is —C(O)R^{x}. In some embodiments, R⁵ is —Ac. In some embodiments, R⁵ is —R^{x}. In some embodiments, R⁵ is —C(O)OR^{y}. In some embodiments, R⁵ is —C(O)N(H)R^{y}. In some embodiments, R⁵ is —(C₂H₅O)ₚR^{y}. In some embodiments, R⁵ is —P(O)(OH)₂. In some embodiments, R⁵ is —S(O₂)OH. In some embodiments, R⁵ is —(CⱼHₖOₘ).

### R^{a}

In some embodiments, R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ), where —(CⱼHₖOₘ) can be in linear or cyclic form. In some embodiments, R^{a} is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R^{a} is —H. In some embodiments, R^{a} is —P(O)(OH)₂, or —S(O₂)OH. In some embodiments, R^{a} is —C(O)R^{x}. In some embodiments, R^{a} is —Ac. In some embodiments, R^{a} is —R^{x}. In some embodiments, R^{a} is —C(O)OR^{y}. In some embodiments, R^{a} is —C(O)N(H)R^{y}. In some embodiments, R^{a} is —(C₂H₅O)ₚR^{y}. In some embodiments, R^{a} is —P(O)(OH)₂. In some embodiments, R^{a} is —S(O₂)OH. In some embodiments, R^{a} is —(CⱼHₖOₘ).

### R^{x}

In some embodiments, each R^{x} is independently C₁-C₃ alkyl, wherein R^{x} is optionally substituted with 1-7 instances of halogen. In some embodiments, R^{x} is methyl, wherein R^{x} is optionally substituted with 1-7 instances of halogen. In some embodiments, R^{x} is ethyl, wherein R^{x} is optionally substituted with 1-7 instances of halogen. In some embodiments, R^{x} is n-propyl, wherein R^{x} is optionally substituted with 1-7 instances of halogen. In some embodiments, R^{x} is i-propyl, wherein R^{x} is optionally substituted with 1-7 instances of halogen. In some embodiments, R^{x} is methyl. In some embodiments, R^{x} is ethyl. In some embodiments, R^{x} is n-propyl. In some embodiments, R^{x} is i-propyl.

### R^{y}

In some embodiments, each R^{y} is independently —H or C₁-C₃ alkyl, wherein R^{y} is optionally substituted with 1-7 instances of halogen. In some embodiments, R^{y} is —H. In some embodiments, each R^{y} is independently C₁-C₃ alkyl, wherein R^{y} is optionally substituted with 1-7 instances of halogen. In some embodiments, R^{y} is methyl, wherein R^{y} is optionally substituted with 1-7 instances of halogen. In some embodiments, R^{y} is ethyl, wherein R^{y} is optionally substituted with 1-7 instances of halogen. In some embodiments, R^{y} is n-propyl, wherein R^{y} is optionally substituted with 1-7 instances of halogen. In some embodiments, R^{y} is i-propyl, wherein R^{y} is optionally substituted with 1-7 instances of halogen. In some embodiments, R^{y} is methyl. In some embodiments, R^{y} is ethyl. In some embodiments, R^{y} is n-propyl. In some embodiments, R^{y} is i-propyl.

In some embodiments, a polyol lipid is a PEFA compound represented by:

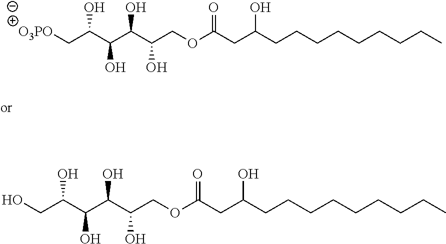

or salt or acid thereof.

### Preparation of Compounds

Below are examples of specific embodiments regarding the invention described by the present disclosure. The examples are offered for illustrative purposes only and are not intended to limit the scope of the present invention in any way. The groups referenced in the examples below, e.g., OR¹, OR², etc., refer to the corresponding groups described herein, e.g., R¹, R², etc., and can be replaced with any of the groups provided in the respective corresponding group, i.e., without the “O.” Efforts have been made to ensure accuracy with respect to numbers used (e.g., amounts, temperatures, etc.), but some experimental error and deviation should, of course, be allowed for.

In some embodiments, compounds of the present disclosure can be prepared as outlined in Scheme 1 or 2:

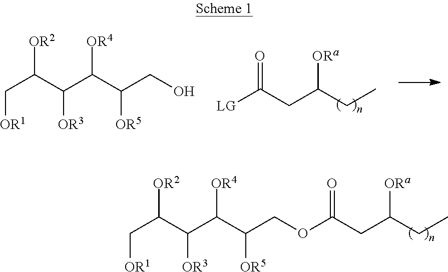

wherein LG is a leaving group.

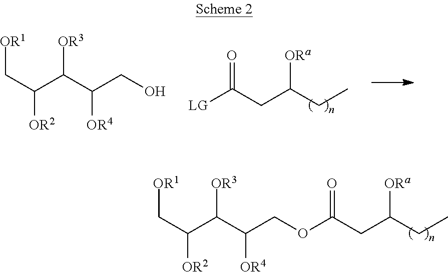

wherein LG is a leaving group.

In some embodiments, compounds of the present disclosure can be prepared as outlined in Scheme 3 or 4:

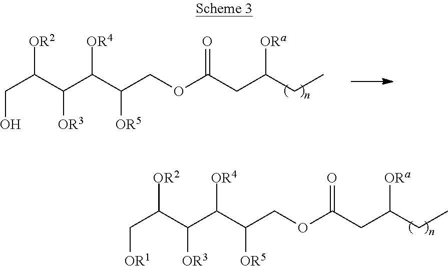

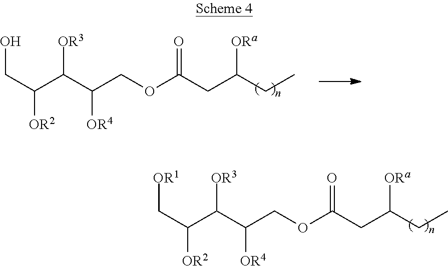

In some embodiments, compounds of the present disclosure can be prepared as outlined in Scheme 5 or 6:

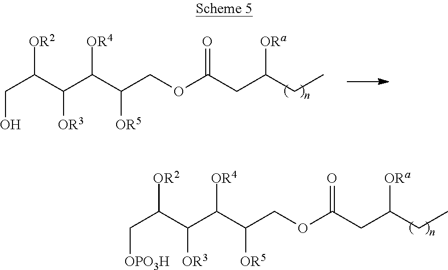

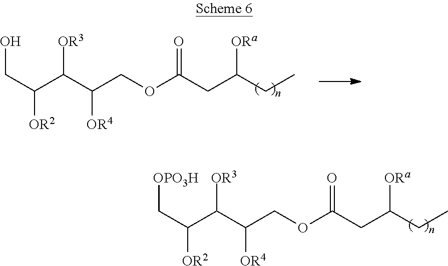

Synthesis/isolation of starting materials for the compounds described herein can be found in WO2018148465 and WO2017184884, each of which are incorporated in their entirety.

A non-limiting example of preparation of Hydroxy-dodecanoic acid 2,3,4,5-tetrahydroxy-6-phosphonooxy-hexyl ester:

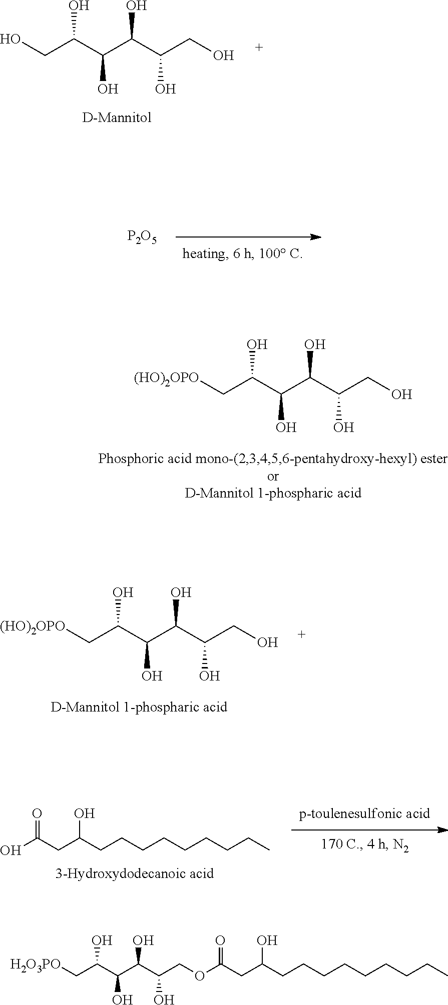

### 3-Hydroxy-dodecanoic acid 2,3,4,5-tetrahydroxy-6-phosphonooxy-hexyl ester

A non-limiting example of preparation of D-Mannitol 1-phosphoric acid: D-mannitol and P₂O₅ are heated neat at 100° C. for 6 hours. As an example, D-Mannitol 1-phosphoric acid can be prepared as outlined in J.-G. Nam et al. Materials Chemistry and Physics 116 (2009) 46-51. D-Mannitol 1-phosphoric acid and 3-hydroxydodecanoic acid are heated in p-toluene sulfonic acid at 170° C. for 4 h at N₂ as outlined in Wenyuan Han et al. Polymers 11, (2019), 1031.

### Emulsifiers

An emulsifier comprising at least one polyol lipid can be used to stabilize water-in-oil emulsions. An emulsifier as described herein can include at least one polyol lipid used to stabilize oil-in-water emulsions.

In some embodiments, an emulsifier is inert so as to not react with the other components of a product or system in which it is introduced. In some embodiments, an emulsifier reacts minimally with the components of a product or system in which it is introduced. In some embodiments, an emulsifier has no adverse effect on a product or system. In some embodiments, an emulsifier has minimal adverse effect on the product or system. In some embodiments, an emulsifier is present in an end product of a processing procedure. In some embodiments, an emulsifier is physiologically safe or has a safety profile that is biologically acceptable. In some embodiments, an emulsifier is biodegradable and/or environmentally safe or environmentally acceptable.

In some embodiments an emulsifier has a HLB value in the range 0-12 to form water-in-oil emulsions. In some embodiments, an emulsifier has a HLB value in the range 6-18 for oil-in-water emulsions. In some embodiments, an emulsifier can be mixed with another emulsifier to stabilize the emulsions in a final product. The additional emulsifier in some embodiments could include a PEFA compound; but in other embodiments, the additional emulsifier could exclude a PEFA compound.

Emulsifiers and water-in-oil emulsions disclosed herein provide advantages over other conventional agents and formulations in that they are renewable, have low-toxicity, and/or have good biodegradability, while still maintaining emulsion stability over time or when subjected to heat, and can improve the constitution of an end consumer product.

An emulsifier can include at least one polyol lipid. In some embodiments, at least one polyol lipid is a PEFA compound. The PEFA compound can include one of the PEFA compounds described herein, including those represented by Formula (I), (II), (I-a) or (II-a).

In some embodiments, an emulsifier can comprise two or more polyol lipids. In some embodiments, the two or more polyol lipids are polyol esters of fatty acids (PEFAs) compounds. In some embodiments, the two or more polyol lipids are selected from PEFA compounds described herein, including those represented by Formula (I), (II), (I-a), or (II-a).

An emulsifier can be formed with oil and water components. In some embodiments, a method can include adding two or more polyol lipids selected from PEFA compounds described herein, including those represented by Formula (I), (II), (I-a), or (II-a).

An emulsifier can be provided in various forms. For example, the emulsifier can be provided in dry form. In some embodiments, an effective amount of an emulsifier can be added to a product mixture to form a stable emulsion. An effective amount depends on the amount of fat or oil that needs to be stably dispersed in the emulsion. An effective amount may also depend on the type of emulsion used. In some embodiments, an effective amount added is about 2-3 times less than that needed when conventional emulsifiers are used. For example, 5 weight percent (wt %) instead of 15 wt % of emulsifier can be provided to emulsify 10 wt % of oil.

In some embodiments, an emulsifier can have pentitol polyol esters of fatty acids and hexitol polyol esters of fatty acids. In some embodiments, the C16 and C18 fatty acid components of the pentitol and hexitol polyol esters can comprise about 20% to about 99% of the PEFA; and in some embodiments from about 30% to about 95% of the PEFA; in some embodiments from about 40% to about 90% of the PEFA. In some embodiments, the C16 and C18 fatty acid components of the pentitol and hexitol polyol esters can comprise about 50% or greater of the PEFA; in some embodiment, about 60% or greater of the PEFA; and in some embodiments, about 70% or greater of the PEFA; and in some embodiments, about 80% or greater of the PEFA. In some embodiments, the ratio of pentitol to hexitol polyol esters in the surfactant is about 5:1 or greater; in some embodiment, the ratio is about 8:1 or greater; in some embodiments, the ratio is about 10:1 or greater; in some embodiments, the ratio is about 15:1 or greater; in some embodiments is about 20:1 or greater; in some embodiments, the ratio is about 30:1 or greater; in some embodiments, the ratio is about 40:1 or greater; in some embodiments is about 50:1 or greater.

### Water-In-Oil Emulsions and Oil-In-Water Emulsions

A water-in-oil emulsion can comprise at least one polyol lipid. In some embodiments, at least one polyol lipid is a PEFA compound. In some embodiments, at least one polyol lipid is selected from PEFA compounds described herein, including those represented by Formula (I), (I), (I-a), or (II-a).

In some embodiments, a water-in-oil emulsion can comprise two or more polyol lipids. In some embodiments, an oil-in-water emulsion comprises two or more polyol lipids. In some embodiments, the two or more polyol lipids are PEFAs. In some embodiments, the two or more polyol lipids are selected from PEFA compounds described herein, including those represented by Formula (I), (II), (I-a), or (II-a).

Water-in-oil emulsions and oil-in-water emulsions can further comprise a solvent, a carrier, an additive, a stabilizing agent, a surfactant, an emulsifier, or a combination thereof.

In some embodiments, a water-in-oil emulsion and an oil-in-water emulsion can each further include a solvent. Such solvents can be selected for dissolving or dispersing the at least one polyol lipid. Suitable solvents include any of a variety of solvents that solubilize but do not significantly degrade at least one polyol lipid.

Non-limiting examples of solvents include hydrocarbons (both aromatic and aliphatic), oxygenated solvents (alcohols, ketones, aldehydes, ethers, glycol ethers, esters, and glycol ether esters), polyalkylene oxide, capped polyalkylene oxide, and combinations thereof. Suitable polyalkylene oxides include polyethylene glycol, polypropylene glycol, polybutylene glycol, mixtures thereof, or the like. Suitable capped polyalkylene oxides include mono-alkyl and di-alkyl ethers of the respective polyalkylene oxides, such as mono- and di-methyl ethers of polyalkylene glycol, mono- and di-ethyl ethers of polyalkylene glycol, mono- and di-propyl ethers of polyalkylene glycol, mono- and di-butyl ethers of polyalkylene glycol, mixtures thereof, or the like. Suitable capped polyalkylene oxides include methyl polyethylene glycol (e.g., the monomethyl ether of polyethylene glycol), dimethyl polyethylene glycol (e.g., the dimethyl ether of polyethylene glycol), mixtures thereof, or the like.

Suitable solvent solubilizers include glycol ethers. Suitable glycol ethers include diethylene glycol n-butyl ether, diethylene glycol n-propyl ether, diethylene glycol ethyl ether, diethylene glycol methyl ether, diethylene glycol t-butyl ether, dipropylene glycol n-butyl ether, dipropylene glycol methyl ether, dipropylene glycol ethyl ether, dipropylene glycol propyl ether, dipropylene glycol tert-butyl ether, ethylene glycol butyl ether, ethylene glycol propyl ether, ethylene glycol ethyl ether, ethylene glycol methyl ether, ethylene glycol methyl ether acetate, propylene glycol n-butyl ether, propylene glycol ethyl ether, propylene glycol methyl ether, propylene glycol n-propyl ether, tripropylene glycol methyl ether and tripropylene glycol n-butyl ether, ethylene glycol phenyl ether (commercially available as DOWANOL EPH™ from Dow Chemical Co.), propylene glycol phenyl ether (commercially available as DOWANOL PPH™ from Dow Chemical Co.), and the like, or mixtures thereof. Additional suitable commercially available glycol ethers (all of which are available from Union Carbide Corp.) include Butoxyethyl PROPASOL™, Butyl CARBITOL™ acetate, Butyl CARBITOL™, Butyl CELLOSOLVE™ acetate, Butyl CELLOSOLVE™, Butyl DIPROPASOL™, Butyl PROPASOL™, CARBITOL™ PM-600, CARBITOL™ Low Gravity, CELLOSOLVE™ acetate, CELLOSOLVE™, Ester EEP™, FILMER IBT™, Hexyl CARBITOL™, Hexyl CELLOSOLVE™, Methyl CARBITOL™, Methyl CELLOSOLVE™ acetate, Methyl CELLOSOLVE™, Methyl DIPROPASOL™, Methyl PROPASOL™ acetate, Methyl PROPASOL™, Propyl CARBITOL™, Propyl CELLOSOLVE™, Propyl DIPROPASOL™ and Propyl PROPASOL™.

A water-in-oil emulsion and an oil-in-water emulsion can each further include a carrier. A carrier can provide a medium which dissolves, suspends, or carries the other components of an emulsion composition. For example, the carrier can provide a medium for solubilization, suspension, or production of polyol lipid and for forming an equilibrium mixture. The carrier can also function to deliver antimicrobial components of an emulsion composition on an object. To this end, the carrier can contain any component or components that can facilitate such delivery and serve additional functions for the emulsion composition.

In certain embodiments, the carrier includes primarily water which can promote solubility and work as a medium for reaction and equilibrium. Non-limiting examples of carriers can include one or more of hydrocarbon oils, water, fatty alcohols and esters, and organic solvents, such as simple alkyl alcohols, e.g., ethanol, isopropanol, n-propanol, and the like.

In certain embodiments, the carrier makes up a large portion of either a water-in-oil emulsion or oil-in-water emulsion. The carrier concentration and type will depend upon the nature of the formulation, the environmental storage, and method of application including concentration of the polyol lipid, among other factors.

In certain embodiments, a water-in-oil emulsion includes about 0 to about 98 wt % carrier, about 0.001 to about 99.99 wt % carrier, about 0.2 to about 60 wt % carrier, about 1 to about 98 wt % carrier, about 5 to about 99.99 wt % carrier, about 5 to about 97 wt % carrier, about 5 to about 90 wt % carrier, about 5 to about 70 wt % carrier, about 5 to about 20 wt % carrier, about 10 to about 90 wt % carrier, about 10 to about 80 wt % carrier, about 10 to about 50 wt % carrier, about 10 to about 20 wt % carrier, about 15 to about 70 wt % carrier, about 15 to about 80 wt % carrier, about 20 to about 70 wt % carrier, about 20 to about 50 wt % carrier, about 20 to about 40 wt % carrier, about 20 to about 30 wt % carrier, about 30 to about 75 wt % carrier, about 30 to about 70 wt % carrier, about 40 to about 99.99 wt % carrier, about 40 to about 90 wt % carrier, or about 60 to about 70 wt % carrier.

In certain embodiments, an oil-in-water emulsion includes about 0 to about 98 wt % carrier, about 0.001 to about 99.99 wt % carrier, about 0.2 to about 60 wt % carrier, about 1 to about 98 wt % carrier, about 5 to about 99.99 wt % carrier, about 5 to about 97 wt % carrier, about 5 to about 90 wt % carrier, about 5 to about 70 wt % carrier, about 5 to about 20 wt % carrier, about 10 to about 90 wt % carrier, about 10 to about 80 wt % carrier, about 10 to about 50 wt % carrier, about 10 to about 20 wt % carrier, about 15 to about 70 wt % carrier, about 15 to about 80 wt % carrier, about 20 to about 70 wt % carrier, about 20 to about 50 wt % carrier, about 20 to about 40 wt % carrier, about 20 to about 30 wt % carrier, about 30 to about 75 wt % carrier, about 30 to about 70 wt % carrier, about 40 to about 99.99 wt % carrier, about 40 to about 90 wt % carrier, or about 60 to about 70 wt % carrier.

In some embodiments, a water-in-oil emulsion and an oil-in-water emulsion can each further include one or more additives. Non-limiting examples of additives include ethylene oxide/propylene oxide block copolymers, butylene oxide/propylene oxide block copolymers, ethylene oxide/butylene oxide block copolymers, waxes, or silicone-based materials.

In some embodiments, a water-in-oil emulsion and an oil-in-water emulsion can each further include a stabilizing agent. The shelf-life of a water-in-oil emulsion or oil-in-water emulsion as disclosed herein can be improved by including a stabilizing agent and/or a preservative to prevent bacterial spoilage. Non-limiting examples of stabilizing agents include oleic acid, hexylene glycol, fatty alcohols, naphthalene sulfonates, butyl alcohol, and formaldehyde.

In some embodiments, a surfactant or secondary emulsifier is selected to be added to the water-in-oil emulsion or oil-in-water emulsion so as to improve the wettability of the emulsion. For example, the improved wettability is useful when such emulsions are applied on a foodstuff, or when such emulsions are formed with at least one polyol lipid. In some embodiments, such surfactants or secondary emulsifiers may be anionic, cationic, nonionic or amphoterics and combination thereof. Non-limiting examples of anionic surfactants or secondary emulsifiers include alkali metal, ammonium and amine soaps; the fatty acid part of such soaps contains preferably at least 16 carbon atoms. Other non-limiting examples of anionic surfactants or secondary emulsifiers include alkali metal salts of alkyl-aryl sulfonic acids, sodium dialkyl sulfosuccinate, sulfated or sulfonated oils, e.g., sulfated castor oil; sulfonated tallow, and alkali salts of short chain petroleum sulfonic acids. Non-limiting examples of cationic surfactants or secondary emulsifiers include salts of long chain primary, secondary or tertiary amines, such as oleylamide acetate, cetylamine acetate, di-dodecylamine lactate, the acetate of aminoethyl-aminoethyl stearamide, dilauroyl triethylene tetramine diacetate, 1-aminoethyl-2-heptadecenyl imidazoline acetate; and quaternary salts, such as cetylpyridinium bromide, hexadecyl ethyl morpholinium chloride, and diethyl di-dodecyl ammonium chloride. Non-limiting examples of nonionic surfactants or secondary emulsifiers include condensation products of higher fatty alcohols with ethylene oxide, such as the reaction product of oleyl alcohol with 10 ethylene oxide units; condensation products of alkylphenols with ethylene oxide, such as the reaction product of isoctylphenol with 12 ethylene oxide units; condensation products of higher fatty acid amides with 5, or more, ethylene oxide units; polyethylene glycol esters of long chain fatty acids, such as tetraethylene glycol monopalmitate, hexaethyleneglycol monolaurate, nonaethyleneglycol monostearate, nonaethyleneglycol dioleate, tridecaethyleneglycol monoarachidate, tricosaethyleneglycol monobehenate, tricosaethyleneglycol dibehenate, polyhydric alcohol partial higher fatty acid esters such as sorbitan tristearate, ethylene oxide condensation products of polyhydric alcohol partial higher fatty acid esters, and their inner anhydrides (mannitol-anhydride, called Mannitan, and sorbitol-anhydride, called Sorbitan), such as glycerol monopalmitate reacted with 10 molecules of ethylene oxide, pentaerythritol monooleate reacted with 12 molecules of ethylene oxide, sorbitan monostearate reacted with 10-15 molecules of ethylene oxide, mannitan monopalmitate reacted with 10-15 molecules of ethylene oxide; long chain polyglycols in which one hydroxyl group is esterified with a higher fatty acid and other hydroxyl group is etherified with a low molecular alcohol, such as methoxypolyethylene glycol 550 monostearate (550 meaning the average molecular weight of the polyglycol ether). Non-limiting examples of amphoteric surfactants include alkybetaine, alkyldimethylamine N-oxide, alkylamidopropylamine N-oxide, alkylamidopropylbetaine, cocamidopropyl betaine, cocoamphoacetate and cocoamphodiacetate, phosphatidylserine, phosphatidylethanolamine, phosphatidylcholine, sphingomyelins, lauryldimethylamine oxide, and myristamine oxide. In some embodiments, a combination of two or more of these surfactants may be used; e.g., a cationic may be blended with a nonionic or an anionic with a nonionic.

One of ordinary skill in the art will appreciate that the individual components of either a water-in-oil emulsion or an oil-in-water emulsion may change depending on the physical and chemical qualities needed for the emulsion stability in a given process and/or application to which the formulation will be applied. For example, the dispersibility of either the water-in-oil emulsion or oil-in-water emulsion in water can be adjusted as necessary to obtain the desired performance.

Hydrophilic-lipophilic balance (HLB) of an emulsion is a measure of the degree to which it is hydrophilic or lipophilic. HLB values are calculated using known methods. In some embodiments, a water-in-oil emulsion disclosed herein has a HLB of 0 to 12. In some embodiments, a water-in-oil emulsion disclosed herein has a HLB of less than 10. In some embodiments, a water-in-oil emulsion disclosed herein is lipid-soluble (water-insoluble). In some embodiments, a water-in-oil emulsion disclosed herein has a HLB of 4 to 10. In some embodiments, a water-in-oil emulsion disclosed herein has a HLB of 6 to 8. In some embodiments, a water-in-oil emulsion disclosed herein has a HLB of 3 to 6.

In some embodiments, an oil-in-water emulsion disclosed herein has a HLB of 6 to 18. In some embodiments, an oil-in-water emulsion disclosed herein has a HLB of less than 18. In some embodiments, an oil-in-water emulsion disclosed herein has a HLB of less than 12. In some embodiments, an oil-in-water emulsion disclosed herein is lipid-soluble (water-insoluble). In some embodiments, an oil-in-water emulsion disclosed herein has a HLB of 6 to 12. In some embodiments, an oil-in-water emulsion disclosed herein has a HLB of 6 to 8. In some embodiments, a polyol lipid has a HLB of less than 12.

As described herein, an emulsifier can be formed from one polyol lipid having a PEFA compound. As a primary emulsifier, the described emulsifier can provide a lowering of interfacial tension to attain a smaller droplet size. This allows the emulsifier to offer effective steric stabilization after a W/O or O/W emulsion is formed. As a primary or sole emulsifier, the described emulsifier can have an HLB value from about 8 to about 10 in certain embodiments; and an HLB value of about 9 in certain embodiments.

The emulsifier described herein can also serve as a co-emulsifier with either another PEFA-based emulsifier or a non-PEFA-based emulsifier. In certain embodiments a PEFA-based emulsifier provides enhanced benefits to an emulsification when it is mixed with another emulsifier that has similar HLB values. For example, each of the co-emulsifiers have HLB values ranging from about 6 to about 12 in certain embodiments; a HLB value from about 8 to about 10 in certain embodiments; and a HLB value of about 9 in certain embodiments.

### Applications

The emulsifiers and related compositions described herein are particularly relevant to food and beverage applications and to cosmetic and dermatologic applications home care, personal care, industrial cleaning, auto care, oil fracking, pharmaceutical, agriculture (e.g., pesticides), paint, adhesives, sealants, coatings, inks, flavors and fragrances.

### Food/Beverage

The methods and processes described herein are effective in allowing immiscible substances, such as oil and water, to mix homogeneously and to produce stable emulsions with a longer shelf life and improved preservation against yeast and mold in the manufacture of various substances, such as manufacturing foodstuffs, as compared to a similar method or process where the polyol esters of fatty acids (PEFAs) described herein are not used. An important consideration in foodstuff production is that the emulsifier used should not impart an off-taste or other negative characteristics to the foodstuff product. Without being limited by theory, it is expected that the methods and processes of the present disclosure have features that (1) provide stable emulsions and/or (2) provide taste neutrality to the foodstuff end product.

In some embodiments, an emulsifier comprising a PEFA compound as described herein can be added to a foodstuff in sufficient quantity to achieve the level stable emulsion necessary for a process. It is recognized that different food processing techniques require varying amounts of emulsifier to achieve the desired result. In some embodiments, the amount of emulsifier added to a food stuff is measured as a percentage of the combined weight of an emulsifier and a foodstuff (total weight of the food composition). In some embodiments, the quantity of the emulsifier is from about 0.001 to 0.5 percent, by weight, of the total weight of the food composition, or about 0.01 to 5 percent, by weight, of the total weight of the food composition. In some embodiments, the quantity of the emulsifier is from about 0.1 to 1 percent, by weight, of the total weight of the food composition. In some embodiments, the quantity of the emulsifier is from about 0.01 to 2 percent, by weight, of the total weight of the food composition, or about 0.01 to about 1 percent, by weight, of the total weight of the food composition.

Nonlimiting examples of foodstuffs where stable emulsion is desirable include: beverages such as flavored carbonated or non-carbonated soft drinks, dairy-based beverages, soy beverages, tea, fruit drinks, flavored juices, sports drinks (e.g., Gatorade®), alcoholic beverages; and non-beverage food products, such as baked goods, syrups, flavor oil emulsions, salad dressings, mayonnaise, dairy products, ice creams, various types of pates, confectionary, jam, and jellies. For example, the emulsifier can be used to stabilize flavor emulsion in beverages such as carbonated soft drinks. Other beverages can also be formed using the emulsifier.

Additional nonlimiting examples of foodstuffs where stable emulsion is desirable include beverages in instant powder mix form, and liquid concentrates which need to be reconstituted with other liquids such as milk, water and juices before consumption.

Non-limiting examples of processes using foodstuffs where stable emulsion is desirable include food processing such as rheologic modifiers in food applications including doughs, pastas.

Non-limiting examples of processes using foodstuffs where stable emulsion is desirable include during industrial processing of sugar beet (such as leading to formation of sugar, syrups, and juices), in processing equipment during washing, cutting, diffusing, carbonizing, and evaporation steps; during industrial processing of potatoes, in processing equipment during washing, cleaning, polishing, and cutting; and industrial fermentation processes, including fermentation.

Additional non-limiting examples of processes include food processing such as cleaning agents for fresh and frozen fruits, vegetables, meats and processed foods, food processing such as rheologic modifiers in food applications including doughs, pastas, and food processing such as antiadherents.

In some embodiments, a food composition or a beverage disclosed herein does not comprise a petroleum-based surfactant.

In some embodiments, a food composition or a beverage disclosed herein, can have one or more improved properties compared to food compositions or beverages comprising conventional emulsifiers. In some embodiments, the one or more improved properties can be selected from the group comprising: increased solubility, improved emulsion capacity, increased stability of emulsified particle, increased stability of emulsified particle size, increased viscosity stability, or improved tactile sensation and/or sensation during use.

In some embodiments, a foodstuff comprises an emulsifier disclosed herein and/or a water-in-oil emulsion disclosed herein and/or an oil-in water emulsion disclosed herein, wherein the emulsifier and/or a water-in-oil emulsion and/or an oil-in water emulsion is in sufficient quantity to achieve emulsion stability over time and emulsion stability when subjected to heat, necessary for a process. It is recognized that different food processing techniques result in varying levels of emulsion stability, and as such, require varying amounts of emulsifiers and/or a water-in-oil emulsions and/or oil-in-water emulsions to achieve the desired result.

### Cosmetic/Dermatologic

Due to skin-friendly properties, the polyol lipids disclosed herein can be used in cosmetic and dermatologic compositions. Possible application forms of cosmetic and dermatologic compositions comprising at least one polyol lipid can include sprays, lotions, creams, ointments and thus use over a very wide consistency range from water-thin to heavily pasty, or solid.

Non-limiting examples of cosmetic and dermatological compositions include creams, foams, mousses, balms, ointments, masks, personal care formulations such as shampoos, body washes, conditioners, soaps, creams, skin treatments, and moisturizing agents.

Additional non-limiting examples of cosmetic and dermatological compositions include care creams and lotions for face, body and hands, in sunscreen emulsions, in make-up, in aerosols, roll-ons, pump sprays, sticks e.g. in the antiperspirant/deodorant sector, in baby care products, in intimate care products, foot care products, hair care products, nail care products, dental care products or oral care products, and also in dermatological ointments.

In some embodiments, a cosmetics and dermatologic composition can include a polyol lipid. Such polyol lipids can further include one or more cosmetic active ingredient. Non-limiting examples of cosmetic or biogenic active ingredients include phytosphingosine (and phytosphingosin derivatives), sphingosine (and sphingosine derivatives), sphingolipids, tocopherol, tocopherol acetate, tocopherol palmitate, ascorbic acid, polyphenols, deoxyribonucleic acid, coenzyme Q10, retinol, AHA acids, amino acids, hyaluronic acid, alpha-hydroxy acids, flavones, isoflavones, stilbenes, catechines, polyglutamic acid, creatine (and creatine derivatives), guanidine (and guanidine derivatives), pseudoceramides, essential oils and fatty acids, peptides, preferably peptides comprising from 2 to 10 amino acids, oligopeptides, protein hydrolysates, plant extracts, bisabolol, allantoin, panthenol, phytantriol, idebenone, liquorice extract, plant extracts, glycyrrhizidine and idebenone, scleroglucan, 3-glucan, santalbic acid and vitamin complexes.

In some embodiments, a cosmetic or dermatological composition disclosed herein does not include a petroleum-based surfactant.

In some embodiments, a cosmetic or dermatological composition disclosed herein, has one or more improved properties compared to conventional agents. In some embodiments, the one or more improved properties selected from the group comprising: increased solubility, improved emulsion capacity, increased stability of emulsified particle, increased stability of emulsified particle size, increased viscosity stability, or improved tactile sensation and/or sensation during use.

In some embodiments, a cosmetic or dermatologic composition comprises an emulsifier disclosed herein and/or a water-in-oil emulsion disclosed herein and/or an oil-in-water emulsion disclosed herein, wherein the emulsifier and/or the water-in-oil emulsion and/or the oil-in-water emulsion is in sufficient quantity to achieve emulsion stability over time and emulsion stability when subjected to heat, necessary for a process. It is recognized that different cosmetic and dermatological compositions require varying levels of emulsion stability, and as such, require varying amounts of emulsifiers and/or water-in-oil emulsions and/or oil-in-water emulsions to achieve the desired result.

In some embodiments, a cosmetic or dermatologic composition comprises an emulsifier disclosed herein and/or a water-in-oil emulsion disclosed herein and/or an oil-in-water emulsion disclosed herein has improved properties compared to conventional agents.

Nonlimiting examples of improved properties include increased solubility, improved emulsion capacity, increased stability of emulsified particle, increased stability of emulsified particle size, improved wetting, improved suspension, increased viscosity stability, and improved tactile sensation and/or sensation during use (e.g., improved feeling to touch, improved feeling of use, sustainably suppress oiliness, maintain moisture retention after application, and not causing stickiness).

### Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

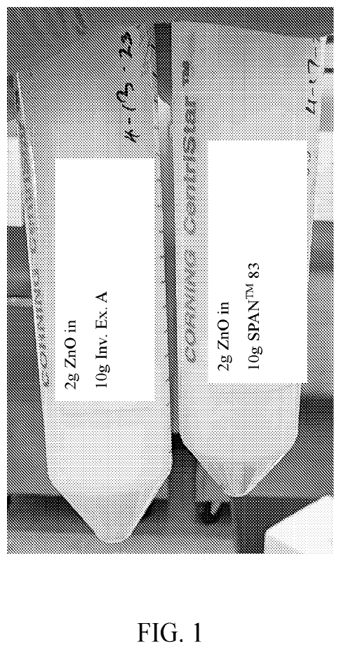

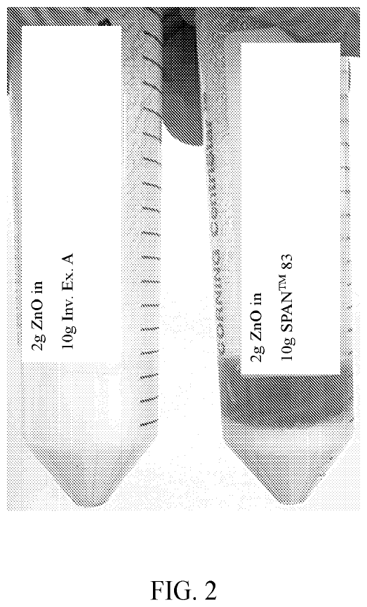

A wetting or dispersant characteristic can be important when identifying an effective emulsifier for cosmetic or dermatological applications. For example, the dispersant characteristic can ensure stability of the composition throughout the manufacturing, storage, and application processes. Such emulsifiers can properly “wet” solid particles by properly replacing the air that surrounds the particles, thereby preventing unwanted lumping, to effectively and uniformly disperse the particles in solution. The emulsifiers described herein provide improved performance when evaluating such wetting or dispersant property characteristics compared to other well-known W/O emulsion compositions. For example, sorbitan sesquioleate (e.g., SPAN™ 83) is a well-known and used W/O emulsion composition for personal care compositions such as those used in cosmetic or dermatological applications. As shown in FIG. 1, the vial on the left (vial 1) contains 20% ZnO particles added to an Inventive Emulsifier A and the vial on the right (vial 2) contains 20% ZnO particles suspended in SPAN™ 83. FIG. 1 illustrates a time period just after the vials have been agitated and both vials illustrate an emulsion system where the ZnO particles are suspended. FIG. 2 shows these same two vials 1 week later where the vials have been stored at 60° C. As can be seen, the ZnO particles remain suspended in vial 1 that contains Inventive Emulsifier A, however, the ZnO particles have settled at the bottom of vial 2 containing the well-known emulsion composition indicating phase separation and instability. These results show the improved performance of Inventive Emulisifier A over that of SPAN™ 83.

In addition, this result illustrates improved stability of Inventive Emulsifier A, as the color of vial 1 has remained white after 1 week, while the composition in vial 2 has darkened and has a yellowish or brownish coloring. Stability is an important feature for compositions employed in personal care compositions and foodstock compositions, namely related to a product's shelf-life and evenly dispersed actives. Thus, the Inventive Emulsifier A is expected to offer improved shelf-life and better sun protection factor (SPF) delivery to any emulsion composition and related products seeking such benefits.

Tables 1-7 provide a sampling of the breadth of applications in which the inventive emulsions described herein can be used. As noted herein, the emulsifiers and related compositions described herein are relevant to a number of industries and can be used in lotions, skin creams, conditioners, facial cleaners, and surface cleaners, among other compositions.

Inventive emulsions described herein can help to provide stability in emulsion compositions, among other benefits. For example, Table 1 shows a formulation of a lotion emulsion composition including Inventive Emulsifier B. Inventive Emulsifier B can lower an interfacial tension to allow for a small droplet size. Further, Inventive Emulsifier B can create a steric barrier around droplets and combine with xanthan gum to provide stability for an emulsion composition for extended room temperature and elevated temperature conditions. Specifically, with Inventive Emulsifier B, the lotion emulsion composition was found to be stable at room temperature, 40° C., and 45° C. for four weeks.

TABLE 1
Formulation for Lotion

| | Wt. | | | | |
|---|---|---|---|---|---|
| Phase | % | Tradename | INCI Name | Supplier | Function |
| A | 62 | Water | Water | | Solvent |
| B | 10 | Zemea ® | Propanediol | Covation | Humectant |
| | | | | Bio | |
| B | 1 | Farmal ® | Xanthan Gum | Ingredion | Thickener |
| | | XANTHAN | | | |
| | | 2312 TC | | | |
| B | 1 | Spectrastat ™ | Caprylhy- | Inolex | Preser- |
| | | | droxamic Acid, | | vative |
| | | | Caprylyl | | |
| | | | Glycol, and | | |
| | | | Glycerin | | |
| B | 1 | SharoSENSE ™ | Polyquater- | Sharon | Preser- |
| | | Plus 181-N | nium-80 and | Labora- | vative |
| | | | Maltol | tories | |
| | | | | Ltd. | |
| C | 20 | C'ESTER MCT | Caprylic/ | Ethox | Emollient |
| | | | Capric | | |
| | | | Triglycer- | | |
| | | | ide | | |
| C | 5 | RubyBio GL | Glycolipids | RubyBio | Emulsifier |
| | | (Inventive | | | |
| | | Emulsifier B) | | | |

In order to formulate the lotion of Table 1, water is heated to 75° C. Separately, Inventive Emulsifier B and the emollient are mixed and then heated to 75° C. Next, the humectant, thickener, and preservatives are mixed together and added to the water while being stirred with an impeller. The mixture of Inventive Emulsifier B and the emollient are then added to the rest of the components while stirring and mixing is continued without heat. The components are homogenized until glossy.

Table 2 shows a formulation of another lotion emulsion composition, which includes Inventive Emulsifier C.

TABLE 2
Formulation for Lotion

| Component | Wt. % |
|---|---|
| Water | 84.81 |
| Glycerin | 3 |
| Sodium Benzoate | 0.5 |
| Isopropyl Palmitate | 2 |
| Cetyl Alcohol | 2 |
| Beeswax | 1 |
| Almond Oil | 4 |
| Citric Acid | 0.2 |
| Fragrance | 0.5 |
| Required Emulsifiers | 0 |
| Tween 80 | 0.91 |
| Inventive Emulsifier C | 1.08 |

Table 3 shows a formulation of a skin cream emulsion composition, which includes Inventive Emulsifier D.

TABLE 3
Formulation for Skin Cream

| Component | Wt. % |
|---|---|
| Water | 65.8 |
| Glycerin | 3 |
| Sodium Benzoate | 1 |
| Isopropyl Palmitate | 15 |
| Cetyl Alcohol | 10 |
| Beeswax | 2 |
| Fragrance | 0.2 |
| Tween 80 | 1.14 |
| Inventive Emulsifier D | 1.86 |

Table 4 shows a formulation of a rinse-out hair conditioner emulsion composition, which includes Inventive Emulsifier E.

TABLE 4
Formulation for Conditioner

| Component | Wt. % |
|---|---|
| Water | 79.93 |
| Quaternized Surfactant | 5.2 |
| Glycerin | 3.1 |
| Guar Gum | 0.52 |
| Citric Acid | 0.05 |
| Coconut Oil | 6 |
| Cetyl Alcohol | 2.1 |
| Fragrance | 0.1 |
| Tween 80 | 1.1 |
| Inventive Emulsifier E | 1.9 |

Table 5 shows a formulation of a leave-in hair conditioner emulsion composition, which includes Inventive Emulsifier F.

TABLE 5
Formulation for Conditioner

| Component | Wt. % |
|---|---|
| Water | 75 |
| Quaternary Surfactant | 2 |
| Glycerin | 3.5 |
| Sodium Benzoate | 1 |
| Isopropyl Palmitate | 5 |
| Cetyl Alcohol | 5 |
| Beeswax | 4 |
| Almond Oil | 1 |
| Guar Gum | 0.5 |
| Fragrance | 0.5 |
| Tween 80 | 0.8 |
| Inventive Emulsifier F | 1.7 |

Table 6 shows a formulation of a surface cleaner emulsion composition, which includes Inventive Emulsifier G.

TABLE 6
Formulation for Surface Cleaner

| Component | Wt. % |
|---|---|
| Water | 97.2 |
| Sodium Lauryl Sulfate | 0.8 |
| Tetrasodium Glutamate Diacetate | 0.2 |
| Sodium Citrate Dihydrate | 0.95 |
| Preservative (MBS) | 0.3 |
| Citric Acid | 0.1 |
| Fragrance | 0.25 |
| Inventive Emulsifier G | 0.2 |

Table 7 shows a formulation of a makeup remover emulsion composition, which includes Inventive Emulsifier H.

TABLE 7
Formulation for Makeup Remover

| Component | Wt. % |
|---|---|
| Isopropyl Myristate | 80.9 |
| Coconut Oil | 4 |
| Olive Oil | 4 |
| Alkyl Polyglycosides | 0.2 |
| Fragrance | 0.1 |
| Tween 80 | 4 |
| Caprylyl Glycol | 0.8 |
| Citric Acid | QS |
| Inventive Emulsifier H | 6 |

### Emulsifier Evaluation

HLD parameters are determined using methods known in the art (e.g., a mixture method). And as noted above, measurement of HLB is also determined according to methods known in the art (e.g., calculation Davies and Lin group contribution method according to Kothencz et al. (2017) Studia Universitatis Babes-bolyai Chemia 62: 451-458).

Those skilled in the art will recognize or be able to ascertain using no more than routine experimentation, many equivalents to the specific embodiments in accordance with the invention described herein. The scope of the present invention is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. An emulsifier comprising one or more polyol lipids, wherein the one or more polyol lipids are selected from a polyol ester of fatty acids (PEFA) compound represented by Formula (I) or (II): 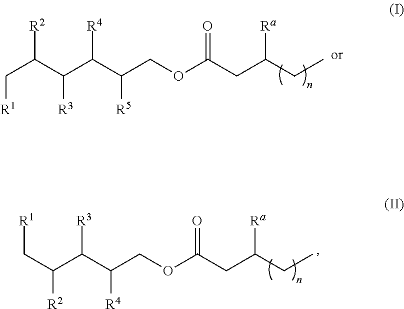
or salt or acid thereof,
wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼH₂ⱼ₋ᵢOⱼ₋₁), —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{x}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
each R^{x} is independently C₁-C₃ alkyl, wherein R^{x} is optionally substituted with 1-7 instances of halogen;
each R^{y} is independently —H, or C₁-C₃ alkyl, wherein R^{x} is optionally substituted with 1-7 instances of halogen;
n is 2-20;
j is 2-12;
k is 4-26;
m is 1-16; and
each p is independently selected from 1-10.

2. An emulsifier comprising one or more polyol lipids, wherein the one or more polyol lipids are selected from:
a) a PEFA compound of Formula (I) or (II): 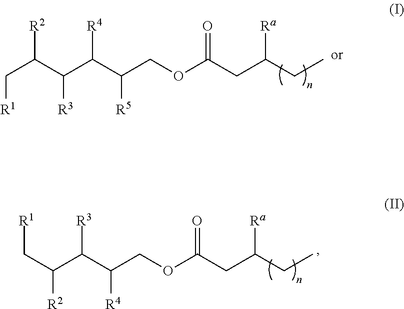
or salt or acid thereof,
wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ));
R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ)ₗ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-20;
j is 2-12;
k is 4-26;
m is 1-16; and
each p is independently selected from 1-10,
wherein
when n is 12 or 14 and R¹ is —OH or —(O)Ac, then R^{a} is not —(O)Ac or —(O)Me;
when n is 12 and R^{a} is —OH, then R² is not —OH or;
when n is 12 or 14, R^{a} is —OH, R² is OH, R³ is OH, and R⁴ is OH, then R¹ is (O)R^{x} or —(O)(C₂H₅O)ₚR^{y};
when n is 12 or 14, R¹ is —(O)S(O₂)OH, R² is —(O)S(O₂)OH, R³ is —(O)S(O₂)OH, R⁴ is —(O)S(O₂)OH, and R⁵ is —(O)S(O₂)OH, then R^{a} is —(O)C(O)R^{x}, R^{x}, —(O)C(O)OR^{x}, —(O)C(O)N(H)R^{x}, —(O)(C₂H₅O)ₚR^{y}, —(O)P(O)(OH)₂, or —(O)S(O₂)OH;
b) the group consisting of an acetylated C12:0-3-hydroxy fatty acid esterified to D-arabitol with 3 acetylations, an acetylated C14:0-3-hydroxy fatty acid esterified to D-mannitol with 4 acetylations, an acetylated C16:0-3-hydroxy fatty acid esterified to D-mannitol with 2 acetylations, an acetylated C16:0-3-hydroxy fatty acid esterified to D-arabitol with 2 acetylations, an acetylated C14:0-3-hydroxy fatty acid esterified to D-arabitol with 4 acetylations, an acetylated C14:0-3-hydroxy fatty acid esterified to D-mannitol with 5 acetylations, an acetylated C16:0-3-hydroxy fatty acid esterified to D-mannitol with 3 acetylations, an acetylated C16:0-3-hydroxy fatty acid esterified to D-arabitol with 3 acetylations, an acetylated C16:0-3-hydroxy fatty acid esterified to D-mannitol with 4 acetylations, an acetylated C18:0-3-hydroxy fatty acid esterified to D-mannitol with 2 acetylations, an acetylated C16:0-3-hydroxy fatty acid esterified to D-mannitol with 5 acetylations, an acetylated C16:0-3-hydroxy fatty acid esterified to D-mannitol with 4 acetylations, an acetylated C18:0-3-hydroxy fatty acid esterified to D-mannitol with 2 acetylations, an acetylated C16:0-3-hydroxy fatty acid esterified to D-mannitol with 5 acetylations, an acetylated C16:0-3-hydroxy fatty acid esterified to D-arabitol with 4 acetylations, an acetylated C18:0-3-hydroxy fatty acid esterified to D-mannitol with 3 acetylations, an acetylated C18:0-3-hydroxy fatty acid esterified to D-mannitol with 4 acetylations, an acetylated C18:0-3-hydroxy fatty acid esterified to D-arabitol with 3 acetylations, an acetylated C18:0-3-hydroxy fatty acid esterified to D-mannitol with 5 acetylations, an acetylated C18:0-3-hydroxy fatty acid esterified to D-arabitol with 4 acetylations, an acetylated C20:0-3-hydroxy fatty acid esterified to D-mannitol with 3 acetylations, an acetylated C20:0-3-hydroxy fatty acid esterified to D-mannitol with 4 acetylations; or
c) the group consisting of: 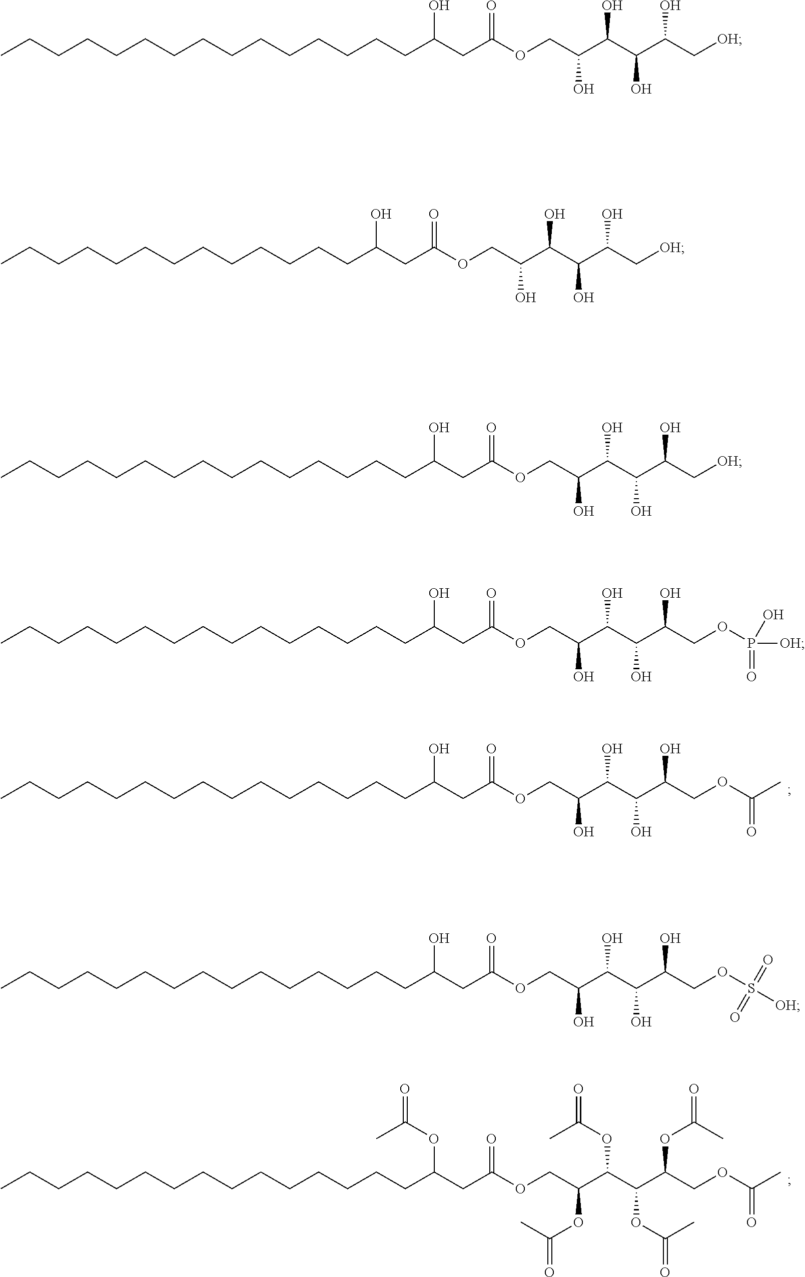 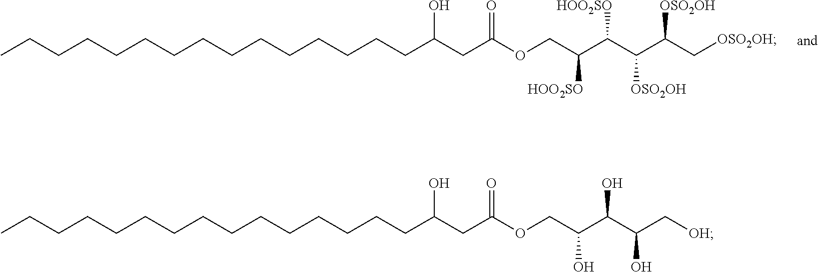
or salt or acid thereof.

3. The emulsifier of any one of claims 1 or 2, wherein the emulsifier comprises two or more of the polyol lipids.

4. The emulsifier of claims 1 or 2, wherein the PEFA compound has an n value from 4 to 20.

5. The emulsifier of claims 1 or 2, wherein the one or more polyol lipids comprise pentitol polyol esters, hexitol polyol esters, or a combination thereof.

6. The emulsifier of claim 5, wherein the ratio of pentitol to hexitol polyol esters is about 5:1 or greater.

7. The emulsifier of claim 5, wherein the ratio of pentitol to hexitol polyol esters is about 20:1 or greater.

8. The emulsifier of claim 5, wherein the C16 and C18 fatty acid components of the pentitol and hexitol polyol esters can comprise about 20% to about 99% of the PEFA compound.

9. The emulsifier of claim 5, wherein the C16 and C18 fatty acid components of the pentitol and hexitol polyol esters can comprise about 30% to about 95% of the PEFA compound.

10. The emulsifier of claim 5, wherein the C16 and C18 fatty acid components of the pentitol and hexitol polyol esters can comprise about 40% or more of the PEFA compound.

11. The emulsifier of claims 1-10, wherein the compound is further represented by Formula (I-a) or (II-a): 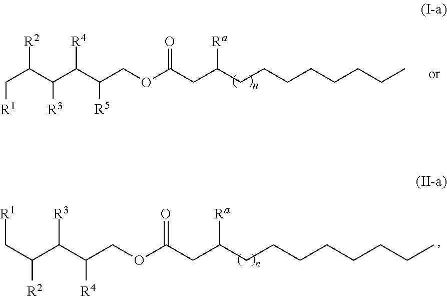
or salt or acid thereof,
wherein
n is 6-12.

12. A personal care composition comprising the emulsifier of claims 1 or 2.

13. The personal care composition of claim 10 is a cosmetic or dermatological composition.

14. A foodstock composition comprising the emulsifier of claims 1 or 2.

15. A water-in-oil emulsion composition comprising an emulsifier of claims 1 or 2.

16. The water-in-oil emulsion composition of claim 15, wherein the emulsifier is a primary emulsifier.

17. The water-in-oil emulsion composition of claim 16 further comprising a secondary emulsifier, wherein the secondary emulsifier is a non-PEFA emulsifier; and wherein the primary emulsifier and secondary emulsifier have similar HLB values.

18. The water-in-oil emulsion composition of claim 17, wherein the HLB values of the primary emulsifier and the secondary emulsifier ranges from about 4 to about 18.

19. An oil-in-water emulsion composition comprising an emulsifier of claims 1 or 2.

20. The oil-in-water emulsion composition of claim 19, wherein the emulsifier is a primary emulsifier.

21. The oil-in-water emulsion composition of claim 20 further comprising a secondary emulsifier, wherein the secondary emulsifier is a non-PEFA emulsifier; and wherein the primary emulsifier and secondary emulsifier have similar HLB values.

22. The oil-in-water emulsion composition of claim 21, wherein the HLB values of the primary emulsifier and the secondary emulsifier ranges from about 6 to about 12.

23. The water-in-oil emulsion composition claim 15, wherein the composition comprises two or more polyol lipids.

24. The oil-in-water emulsion composition of claim 18, wherein the composition comprises two or more polyol lipids.

25. A cosmetic or dermatological composition comprising an emulsifier of claims 1 or 2, wherein the at least one polyol lipid comprises an HLB value of less than 12.

26. The cosmetic or dermatological composition of claim 25 is in the form of a water-in-oil emulsion or an oil-in-water emulsion.

27. The cosmetic or dermatological composition of claim 25 does not comprise a petroleum-based surfactant.

28. A food composition comprising a foodstuff and an emulsifier of claim 1 or 2.

29. The food composition of claim 28 comprising two or more polyol lipids.

30. A beverage comprising an emulsifier of claims 1 or 2.

31. The beverage of claim 30, wherein the emulsifier comprises two or more polyol lipids.

32. An agricultural composition comprising an emulsifier of claim 1 or 2.

33. The agricultural composition of claim 32 comprising two or more polyol lipids.

34. A paint composition comprising an emulsifier of claim 1 or 2.

35. The paint composition of claim 34 comprising two or more polyol lipids.

36. A coating composition comprising an emulsifier of claim 1 or 2.

37. The coating composition of claim 36 comprising two or more polyol lipids.

38. An adhesive composition comprising an emulsifier of claim 1 or 2.

39. The adhesive composition of claim 38 comprising two or more polyol lipids.

40. A sealant composition comprising an emulsifier of claim 1 or 2.

41. The sealant composition of claim 40 comprising two or more polyol lipids.
